(19) United States
(12) Patent Application Publication
(10) Pub. No.: US 2013/0023013 A1
(43) Pub. Date: Jan. 24, 2013

(54) METHOD FOR OBTAINING A FERMENTABLE PRODUCT FROM POLLUTED LIGNO-CELLULOSIC BIOMASS

(71) Applicants: Frederic Bataille, Sevres-Anxaumont (FR); Alexandre Briand, Saint Cyr (FR); Stephane Soum, Migne-Auxances (FR)

(72) Inventors: Frederic Bataille, Sevres-Anxaumont (FR); Alexandre Briand, Saint Cyr (FR); Stephane Soum, Migne-Auxances (FR)

(73) Assignee: VALAGRO CARBONE RENOUVELABLE POITOU-CHARENTES, Poitiers (FR)

(21) Appl. No.: 13/638,211

(22) PCT Filed: Mar. 28, 2011
(86) PCT No.: PCT/FR11/50664

§ 371 Date: Sep. 28, 2012

(30) Foreign Application Priority Data
Mar. 29, 2010 (FR) 10 52286

(51) Int. Cl. C12P 19/14; C12P 7/62; C12P 7/28; C12P 7/14; C12P 7/16

(52) U.S. Cl. 435/99; 435/162; 435/160; 435/150; 435/135

(57) ABSTRACT

A method for obtaining a fermentable product intended for the production of metabolites by fermentation, includes the following steps: a) detoxification of a polluted ligno-cellulosic raw material including at least one polluting component; solid-liquid extraction in order to extract the polluting component(s) from the ligno-cellulosic raw material by solubilisation using at least one solvent; recovery of the cleaned ligno-cellulosic raw material; b) pre-treatment of the cleaned ligno-cellulosic raw material by defibration; c) enzymatic hydrolysis of the pre-treated ligno-cellulosic raw material in the presence of cellulose, and obtaining a fermentable product; d) optional purification of the resulting fermentable product. The use of the resulting fermentable product for producing metabolites by fermentation, as well as to a specific method for producing ethanol from the fermentable product are also described.

This invention relates to a method for obtaining, starting from fouled lignocellulosic biomass, a product that is intended for the production of metabolites by fermentation.

The invention also relates to the use of the product that is obtained for producing metabolites by fermentation, in particular ethanol, as well as a particular method for manufacturing ethanol.

The lignocellulosic biomass essentially consists of polysaccharides of cellulose and hemicelluloses, strongly linked to a lignin network by covalent bond and hydrogen bond. These lignocellulosic compounds comprise in particular wood, straw, plants originating from dedicated crops, co-products of agriculture, the forest, or the industry for transformation of wood, collected plant wastes, and industrial or community waste.

It is known that the lignocellulosic substrates can be upgraded for manufacturing different derivative products. The biomass is used in particular for alcohol production, in particular for the production of ethanol or bioethanol, and is then usable as biofuel or ecofuel.

Currently, the existing methods make it possible to use only certain lignocellulosic raw materials.

Among these first raw materials, it is possible to cite in particular grains, such as wheat or corn, or beets. However, the use of this type of resources exhibits significant drawbacks both in terms of yield and because they compete directly with food cultivation and ecosystems. In addition, the production capacity of raw materials is limited compared to the very significant quantities of fuels that are consumed, for example.

Other resources have also been proposed, such as wood, for example, but the known methods are not satisfactory in terms of yield and require the use of concentrated products. In addition, these methods require a stage for chemical pretreatment of the lignocellulosic biomass for hydrolyzing the hemicelluloses and making cellulose accessible, which can generate degraded sugar inhibitors, such as hydroxymethylfurfural.

Furthermore, it proves necessary to use new raw materials. In particular, it would be especially advantageous to be able to employ used lignocellulosic biomasses to recycle them, in particular lignocellulosic biomasses that are considered to be dangerous waste, whose storage and elimination are subject to the ICPE (Installations Classified for the Protection of the Environment) classification. These fouled or treated lignocellulosic substrates are generally substrates that are impregnated with one or more dangerous substance(s), for example varnish, glue, creosote, CCA (copper, chromium and arsenic), CCB (copper, chromium and boron), fireproof agent, fungicide, or insecticide, such as recycled-wood posts, railway ties, treated woods and sawdust, etc.

Today, however, these raw materials cannot be used, because in addition to the drawbacks already mentioned for the methods for treating conventional lignocellulosic raw materials, if the biomass that is used is fouled or treated, substances that inhibit fermentation can be found in the sweet juice and can prevent its use for the production of metabolites by fermentation.

This is why the objective of this invention is to remedy the different drawbacks of the prior art by proposing an effective and economical method that makes it possible to obtain a fermentable product from a fouled or treated lignocellulosic biomass.

For this purpose, the invention has as its object a method for obtaining a fermentable product that is intended for the production of metabolites by fermentation, characterized in that it comprises the following stages:

a) Detoxification of a polluted lignocellulosic raw material that comprises at least one polluting component:
Solid-liquid extraction for extracting by solubilization the polluting component(s) of the lignocellulosic raw material using at least one solvent,
Recovery of the cleaned lignocellulosic raw material,

b) Defibration pretreatment of the lignocellulosic raw material,

c) Treatment by enzymatic hydrolysis of the pretreated lignocellulosic raw material in the presence of cellulase, and production of a fermentable product,

d) Optionally, purification of the fermentable product that is obtained.

Another object of the invention relates to the use of the fermentable product that is obtained, for the production of metabolites by fermentation, in particular for the production of ethanol, butanol, acetone, polyhydroxyalkanoate, and xanthan.

Finally, the purpose of the invention is also a particular method for the production of ethanol from the fermentable product that is obtained.

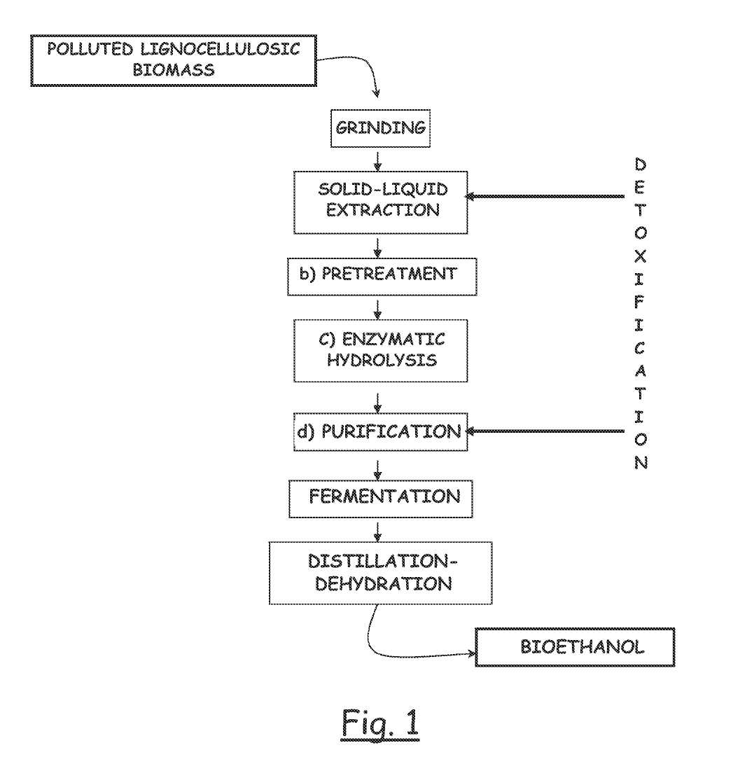

The invention is now described in detail relative to the attached FIG. 1 on which is shown a diagram of a particular embodiment of the method for obtaining a fermentable product from the fouled lignocellulosic biomass.

The object of the invention is therefore a method for obtaining, starting from a polluted lignocellulosic biomass, a fermentable product that can be fermented for producing metabolites.

Within the meaning of the invention, fermentable product is defined as the product based on sugars or sweet juice obtained after enzymatic hydrolysis of the pretreated lignocellulosic material, able to produce metabolites by fermentation.

Likewise, raw material, biomass, or polluted, fouled or treated lignocellulosic substance, within the meaning of the invention, is defined as any lignocellulosic compound that contains at least one molecule that is not found in said compound in the natural state. This molecule is called polluting component in this application. It generally involves a molecule that is toxic or dangerous to humans or the environment.

The polluted lignocellulosic raw material that is used for the implementation of the invention can be obtained from industrial waste, such as textile cotton, paper, products at the end of their service life (pallets, cross-ties of railroad tracks, etc.), wood scraps or sawdust, or scraps of pressboard panels. Preferably, the lignocellulosic raw material that is used is selected from among:
Wood that is treated with polycyclic aromatic hydrocarbons (HAP), i.e., creosote (electrical and telephone poles, railway cross-ties),
Wood treated with copper sulfate or with metal salts (CCA and to a lesser extent CCB),
Wood and sawdust impregnated with oil, grease, paint, . . .
Wood for fences, for construction (frameworks, shutters, gates), for disposable pallets that are treated to increase their durability (pentachlorophenol, creosote, dichlofluanid, propiconazole, isothiazolone, lindane),
Particle board,
Cellulose fibers preserved by pentachlorophenol (military textiles).

According to a first aspect, the method for obtaining a fermentable product that is intended for the production of metabolites by fermentation comprises the following stages according to the invention:

a) Detoxification of a polluted lignocellulosic raw material that comprises at least one polluting component,

b) Pretreatment of the cleaned lignocellulosic raw material,

c) Treatment by enzymatic hydrolysis of the pretreated lignocellulosic raw material in the presence of cellulase, and production of a fermentable product, and

d) Optionally, purification of the fermentable product that is obtained.

Stage a) has as its objective to clean the lignocellulosic raw material of one or more polluting component(s) that it contains.

This first stage comprises the following sub-stages:
Solid-liquid extraction, for extracting by solubilization the polluting component(s) from the lignocellulosic raw material that may or may not be ground, using at least one solvent, and
Recovery of the cleaned lignocellulosic raw material.

The solid-liquid extraction is a technique that is well known to one skilled in the art. In the present case, this extraction consists in extracting one or more soluble component(s) contained in the lignocellulosic raw material using one or more solvent(s) for which the component(s) has/have an affinity.

The solvent(s) used for this stage can be selected in particular from among: water, acetone, acetonitrile, ethanol or ethyl acetate. The selection of solvent depends on the components that are to be eliminated. Ethanol will be selected, for example, to extract polycyclic aromatic hydrocarbons.

Very preferably, the solid-liquid extraction stage is carried out at a temperature of between 50° C. and the boiling point of the solvent, or between 50° C. and the boiling point of the mixture of solvents when there are several solvents.

The solid-liquid extraction is either intermittent (for example, hot maceration), or continuous (for example, Soxhlet, percolation).

At the end of this stage, a liquid phase is recovered that contains solvent(s) and (a) dissolved polluting component(s), and a solid phase that corresponds to the cleaned starting lignocellulosic material, i.e., no longer containing said component(s) or of a small enough quantity to be compatible with the subsequent stages, in particular for being compatible with fermentation.

This detoxification stage makes it possible to clean the majority, and even all, of the lignocellulosic raw material.

The pretreatment stage b) has as its objective to make cellulose accessible for facilitating its enzymatic hydrolysis. This stage consists in carrying out defibration of the lignocellulosic raw material by any suitable method.

This defibration makes it possible to separate the fibers and to increase the digestibility of the cellulose.

Preferably, the cleaned lignocellulosic raw material is defibrated in an extruder, at a temperature of between 60° C. and 180° C., and in the presence of a quantity of water representing between 50% and 450% of the mass of said raw material. The extruder can be a twin-screw or single-screw extruder.

The defibration can be done in the presence of a strong acid or a strong base at a concentration of between 0.5% and 4% relative to the volume of water.

Advantageously, the pretreatment of the raw material by defibration in an extruder makes it possible to increase the yield of the enzymatic hydrolysis of the cellulose. It also makes it possible to reduce the quantity of enzyme used during stage c).

According to a particularly suitable embodiment, the detoxification stage a) is preceded by a preliminary grinding stage of the polluted lignocellulosic raw material that makes it possible to make cellulose even more accessible for the purpose of its hydrolysis. This grinding stage can be carried out by using, for example, a cutting mill.

Stage c) consists in hydrolyzing the cellulose using enzymes to obtain a glucose-rich solution.

Hydrolysis can be done using a cellulase or an enzymatic cocktail that consists of cellulase and β-glucosidase.

Preferably, hydrolysis is done in a tank that is kept at a temperature of between 50° C. and 60° C., and at a pH of between 4 and 5.5 for at least 24 hours. The ratio of enzymes per gram of cellulose is between 0.05 and 2.5.

A fermentable product or a glucose-rich sweet juice is then obtained.

If this product that is obtained also contains one or more residual polluting components that it is desired to eliminate, a purification stage d) is, however, to be implemented before it is possible to have a usable fermentable product.

This second detoxification stage, final detoxification stage, is preferably carried out by adsorption on ion exchange resin or on activated carbon. This adsorption can be carried out in batch mode or on a column, with or without pressure, preferably at ambient temperature and even more preferably by heating between 30° C. and 70° C., particularly between 40° C. and 60° C.

The product that is obtained, free of pollutants, is fermentable with different metabolites.

According to a second aspect, the purpose of the invention is therefore the use of a fermentable product that is obtained for producing metabolites by fermentation.

In terms of the invention, metabolites are defined as the products that are obtained from the transformation of sweet juice by a microorganism.

It may involve in particular a metabolite that is selected from among ethanol, butanol, acetone, polyhydroxyalkanoate, and xanthan.

These metabolites can be obtained by the implementation of fermentation methods that are known by one skilled in the art.

By way of example, the invention proposes a particular method that makes it possible to obtain ethanol from the fermentable product. This particular example of the method according to the invention is illustrated in FIG. 1.

The method for production of ethanol can comprise the series of the following stages:
Implementation of the method that makes it possible to obtain the fermentable product according to the invention,
Fermentation of the product that is obtained, and
Distillation/dehydration of the product that is obtained after fermentation.

According to a variant, the method also comprises a filtration stage before the fermentation stage. This stage can comprise the following sub-stages:
Filtration for the clearance of the medium,
Ultrafiltration,
Reverse osmosis,
Purification, and/or
Sterile filtration.

After the enzymatic hydrolysis, optionally followed by the purification stage, the reaction medium consists of a sweet juice, cellulases, non-cellulosic compounds, and non-degraded cellulose.

A stage of centrifuging, decanting, or prefiltration prior to the filtration makes it possible to eliminate non-cellulosic compounds and non-degraded cellulose.

The ultrafiltration has as its objective to recycle and to reuse the enzymes that are used for hydrolysis.

This tangential ultrafiltration stage makes it possible to considerably reduce the production costs of ethanol, because the cost of enzymes used for the hydrolysis of the cellulose is very high.

The ultrafiltration stage can be carried out on a membrane that makes it possible to concentrate and to recover the enzymes, on the one hand (the concentrate), and the sweet juice, on the other hand (the permeate).

The reverse osmosis concentrates the permeate without evaporation. This stage makes possible an energy savings relative to a conventional evaporation stage.

The color removal makes it possible to retain the dyes of the textile cotton in a resin.

It can be carried out on a column using a resin that has an affinity for the aromatic cycles, in such a way that the dyes of the textile cotton remain in the column by affinity.

The sterile filtration prevents any contamination in the fermenter and ensures an optimum yield of the fermentation.

The fermentation stage has as its objective to ferment in ethanol the glucose that is contained in the filtered sweet juice by means of glycolysis using yeast.

The fermentation can be done in a fermenter at between 30° C. and 37° C., for between 7 hours and 24 hours, and at a pH of between 3.8 and 5.0. Conventionally, the yeasts that are used can be Saccharomyces cerevisiae.

The yield of the fermentation reaches 0.4 g of ethanol per gram of glucose. The yeasts used for the fermentation can be recycled by microfiltration to be reused.

The fermentation stage is generally followed by a distillation/dehydration stage on a membrane that makes it possible to obtain an ethanol at 99.9% that can next be used as a biofuel or agrofuel.

The invention is not, of course, limited to this particular example, and it makes it possible to produce—starting from fouled lignocellulosic raw material—any metabolite that can be obtained by fermentation.

1. Method for obtaining a fermentable product that is intended for the production of metabolites by fermentation, characterized in that it comprises the following stages:
a) Detoxification of a polluted lignocellulosic raw material that comprises at least one polluting component:
Solid-liquid extraction for extracting by solubilization the polluting
component(s) of the lignocellulosic raw material using at least one solvent,
Recovery of the cleaned lignocellulosic raw material,
b) Defibration pretreatment of the cleaned lignocellulosic raw material,
c) Enzymatic hydrolysis of the pretreated lignocellulosic raw material in the presence of cellulase, and production of a fermentable product,
d) Optionally, purification of the fermentable product that is obtained.

2. Method for obtaining a fermentable product according to claim 1, wherein the solid-liquid extraction stage is carried out at a temperature of between 50° C. and the boiling point of the solvent or between 50° C. and the boiling point of the mixture of solvents when there are several solvents.

3. Method for obtaining a fermentable product according to claim 1, wherein the solvent(s) used for the solid-liquid extraction stage is/are selected from among: water, acetone, acetonitrile, ethanol or ethyl acetate.

4. Method for obtaining a fermentable product according to claim 1, wherein the purification stage is carried out by adsorption on ion exchange resin or on activated carbon.

5. Method for obtaining a fermentable product according to claim 4, wherein the purification stage is carried out at a temperature of between 30 and 70° C.

6. Method for obtaining a fermentable product according to claim 1, wherein the detoxification stage a) is preceded by a stage for grinding the lignocellulosic raw material.

7. Method for obtaining a fermentable product according to claim 1, wherein the lignocellulosic raw material is selected from among:
Woods that are treated with polycyclic aromatic hydrocarbons,
Woods that are treated with copper sulfate or with other metal salts,
Woods and sawdust that are impregnated with oil, grease, varnish, or paint,
Woods that are treated for fences, frameworks, shutters, gates, or for disposable pallets,
Particle board,
The cellulosic fibers that are preserved by pentachlorophenol, such as military textiles.

8. Method for obtaining a fermentable product according to claim 1, wherein the defibration of the cleaned cellulosic material is carried out in an extruder.

9. Method for obtaining a fermentable product according to claim 1, wherein the defibration of the cleaned cellulosic material is carried out in a single-screw or twin-screw extruder at a temperature of between 60° C. and 180° C. and in the presence of a quantity of water that represents between 50% and 450% of the mass of said raw material.

10. Method for obtaining a fermentable product according to claim 1, wherein the enzymatic hydrolysis stage is carried out in the presence of cellulase and β-glucosidase.

11. Process for producing a metabolite by fermentation which comprises:
carrying out the method of claim 1 so as to obtain a fermentable product;
subjecting the obtained product to fermentation to obtain a fermented product; and
distilling/dehydrating the fermented product to produce the metabolite.

12. Process according to claim 11, wherein the metabolite is selected from the group consisting essentially of ethanol, butanol, acetone, polyhydroxyalkanoate, and xanthan.

13. Method for the production of ethanol, wherein it comprises the series of the following stages:
Implementation of the method according to claim 1 in such a way as to obtain a fermentable product,
Fermentation of the product that is obtained, and
Distillation/dehydration of the product that is obtained after fermentation.
